(19) United States
(12) Patent Application Publication
(10) Pub. No.: US 2004/0220139 A1
(43) Pub. Date: Nov. 4, 2004

(54) Inhibiting viral infections

(76) Inventor: Nicholas A. Sceusa, New York, NY (US)

Correspondence Address: BROWDY AND NEIMARK, P.L.L.C., 624 NINTH STREET, NW, SUITE 300, WASHINGTON, DC, 20001-5303, US

(21) Appl. No.: 10/700,886

(22) Filed: Nov. 5, 2003

Related U.S. Application Data
(60) Provisional application No. 60/466,043, filed on Apr. 29, 2003.

(51) Int. Cl. A61K 31/739

(52) U.S. Cl. 514/54

(57) ABSTRACT

An antiviral agent administered using Teorell-Meyer gradients is described. The antiviral agent interferes with viral attachment of cells.

### CROSS-REFRERNCE TO RELATED APPLICATIONS

This application claims priority from provisional application No. 60/466,043, filed Apr. 29, 2003, the entire contents of which are hereby incorporated by reference.

### FIELD OF THE INVENTION

The present invention relates to methods and compositions for inhibiting viral infections. More particularly, this invention relates to an antiviral agent that exploits the pH gradient present in skin and mucosal compartments and is delivered to the site where needed by electrostatic forces, and then interferes with the virus' attachment to a cell.

### BACKGROUND OF THE INVENTION

Diseases caused by members of several families of viruses including parainfluenza viruses, rhinoviruses, respiratory syncytial viruses, enteroviruses, and coronaviruses cause much human misery. Yet progress on the treatment of virally-induced diseases has been slow. While there is now a large number of compounds which have been found to exhibit antiviral activity against viruses in culture, antiviral compounds have had limited effectiveness in patients.

Entry sites for viruses include special cells of the mucous membranes. In some cases, viruses may enter during unprotected sexual intercourse. The respiratory tract can be infected via droplet transmission or direct contact, and the digestive tract may be infected by ingestion of infected food, water, or wastewater. Many viruses may also enter the body via the skin.

For a virus to infect a host cell, that cell must have the receptors for the virus on its surface, and the cell also must be capable of supporting viral replication. The host cell receptors are normal surface molecules involved in routine cellular functions. However, since at least a portion of viral receptor site on the viral surface is complementary to the chemical shape of the cell surface molecule that would normally bind to the normal physiological receptor, the virus is able to attach to the host cell's surface. These cell surface molecules are called adhesion molecules or viral receptor sites.

Cell surface receptors are very varied in their chemistry, but are known to be retained by families of viruses, each family, or clan having the same or similar receptor. Examples of adhesion molecules are: glycoproteins, which result in very specific attachment, or carbohydrates or glycolipids found on many surface proteins, which result in less specific attachment, Aminopeptidase N, The Major and Minor Histocompatibility Complexes, and other cell surface constituents. (See Norkin, Leonard C. Virus Receptors: Implications for Pathogenesis and Design of Antiviral Agents, Clinical Microbiological Reviews, April 1995 ppg 293-315, to include the paper's references, all of which are incorporated herein by reference.

Coronaviruses, such as those that cause the common cold, usually attach to cell surfaces or adhesion molecules which are positively charged. Other viruses attach to cell surface receptors which are negatively charged. Sometimes a helper molecule may be involved. Rarely, a mutation may be involved which will change the polarity of the viral attachment site.

The attachment is cell and tissue type specific. For example, rhinoviruses attach to cells in the human upper respiratory tract, specifically to intercellular adhesion molecule (ICAM-1) molecules found on cells of the nasal epithelium. The human immunodeficiency virus (HIV) binds to molecules and receptors on the surface of human T-lymphocytes and macrophages, while herpesviruses bind to viral receptor sites of nerve cells. Pinocytosis or Endocytosis may be involved in some cases; Semliki Forrest Virus, is an example. Thus, there is a need in the art for an antiviral agent.

### SUMMARY OF THE INVENTION

It is an object of the present invention to overcome the aforesaid deficiencies in the prior art.

It is another object of the invention to provide an antiviral agent.

It is another object of the present invention to inhibit viral infections.

It is a further object of the present invention to inhibit viral infection by using a molecule resembling the virus' natural attachment site.

According to the present invention, viral infection is inhibited by blocking viral attachment to cells, thus interfering with viral survival and replication.

For coronaviruses, whose attachment sites are most often Aminopeptidase N, and less often MHC I, the attachment sites can be inhibited by administering ionic Zn²⁺, Cu²⁺, Fe²⁺, Fe³⁺, Al³⁺. However, attachment of a coronavirus can be inhibited by using any suitable sized positively charged molecule. In addition to the specific compounds described above, attachment of coronavirus can be inhibited by positively charged amino acids of suitable configuration, and small positively charged peptides, particularly di- and tri-peptides of correct size, shape and charge.

In the case where the attachment site of the virus is negative, positively charged molecules can be administered to inhibit viral attachment. Suitable inhibitors for these viruses include positively charged ions such as Zn²⁺, Cu²⁺, Al³⁺, Fe²⁺, and Fe³⁺. The ion must have a valence to bind both sides of the site to act as a substrate.

Conversely, for positively charged viral attachement sites, suitable inhibitors for these viruses include negatively charged ions such as: iodine, bromine chlorine, sialic acid, neuraminic acid, acetyl-neuraminic acid, and negatively charged amino acids, and small peptides, particularly di- and tri-peptides of appropriate size and shape, and charge.

In order for the antiviral agent to inhibit replication of the virus, the antiviral agent must be delivered directly to the affected cells. Delivering the antiviral agent orally does not ensure that sufficient antiviral agent reaches the cells affected by the virus. The antiviral agents can be effectively delivered to the affected cells using charge as a driving principle. This method of delivery is described in detail in Sceusa, U.S. Pat. No. 6,414,033, the entire contents of which are hereby incorporated by reference.

### DETAILED DESCRIPTION OF THE INVENTION

A new antiviral agent as well as a method for treating viral infections is disclosed. Said antiviral agent is administered by exploiting naturally occurring concentration gradients. By manipulating the pH of the antiviral agents to a suitable extent, by using a dosage form buffered at a correct pH, the antiviral agent will be moved electro-osmotically in accordance with Teorell-Meyer flux gradients, and thus can be delivered more reliably to the cells affected by the virus.

The design of antiviral agents according to this invention that are capable of moving in a pH dependent manner, derives mathematically from the Teorell-Meyer Theory. See, Teorell, T., Discussions Faraday Soc., 1956, 21(9), 305-369. The derivation according to this invention predicts that a dosage form buffered at the correct pH will be able to move either the desired positive or negative ions from compartment A to compartment B in an pH dependent osmo-electrophoretic manner, provided a flux gradient exists between two compartments.

Such compartments include, without limitation, cell compartments of the human body, such as from the mouth into the naso-pharyngeal area or into the lung; vagina—uterus—Fallopian tubes; outer and inner ear; and many others that are described in the work of Nordenstrom, B. E., Biologically Closed Electrical Systems: Clinical, Experimental and Theoretical Evidence of an Additional Circulatory System; Stockholm, Nordic Medical Publications, 1983, and Evans, E. E., Schentag J. J., Jusko W. J. eds., Applied Pharmacokinetics: Principles of Therapeutic Drug Monitoring, 3rd ed, Vancouver, Wash., 1992, which are incorporated herein by reference, or any standard anatomy and histology text. Other examples of such contiguous compartments include; trachea-bronchioles and bronchi; the surface of the eye; the sclera, the cornea, the anterior chamber, iris, posterior chamber, retina and possibly the optic nerve; the middle and inner ear; and epidural space-meninges-brain, to name a few. The method is also applicable to solid organs as well, such as the liver and prostate. This invention is not limited in scope to the few compartment systems or organs listed here, but is meant to include any such compartment system as meets the basic requirements described herein.

Teorell-Meyer dosage forms depend upon bioelectricity for their function. A biologically closed electric circuit (BCEC) is physiologically analogous to an ordinary electric circuit, except that ions as well as electrons, move along and through the circuit. In biological material, the co-transport of electrons occurs in short redox steps. Ions are transported electro-osmotically. Concentration, and consequently, electrical gradients, are maintained by Donnan Equilibria, large sheets of charge in the tissue proteins, and by ion pumps functioning at the expense of ATP. The second half of the circuit, the return half, takes place via passive or facilitated diffusion. Ions will follow, or respond to the flow of current according to their net charge, from one are of charge density to another area of different charge density, as part of the usual BCEC circulation. The local viscosity, and the electrical path length, which is a vector quantity, plays an important role. Vectors have the properties of force, distance (length), according to the gradients that compose them. Controlling the electrical vector makes it possible to control the ion, because the electrical vector is very many times stronger than any of the other vectors which act.

Although a BCEC is electrically closed, it is thermodynamically and physiologically open, which makes it possible to place a dosage form in a predetermined location. This property is used to artificially induce a gradient, using appropriate buffering, companion, and carrier molecules. Certain molecules may act as all three at the same time, and the amino acids and their congeners are ideal for this purpose. By introducing the specially designed and buffered dosage form, the pH of the recipient compartment, in which the form is placed is changed relative to the target compartment, setting up the induced gradient and corresponding concentration cell. This is provided for by the Lewis acid-base definition, which considers all positive charges as acids and all negative charges as bases.

Inducing the pH change and controlling the bioelectrical field and corresponding electrical vector makes it possible to manipulate the direction of ionic flow and transport. Since the electrical vector is many times more powerful than the other vectors acting, the ionic flow can be stopped or reversed for the time the induced field is present. If the electrical vector is coupled to act in the same direction as the other vectors, the effect is most powerful. The three vectors which are known to act are the hydrostatic vector, the particulate (colligative) vector, and the electro-motive force (electro-osmotic) vector.

It should be remembered that the association constant (Ka) and its reciprocal, the dissolution constant, Kd, for any complex are pH dependent. In the context of an electrical gradient inside a concentration cell, these constants may also be considered to be electrically dependent. This means that at one pH, a complex may be completely associated, and, at another pH, almost completely dissociated.

Therefore, for any given complex, the concentration cell has a continually changing spectrum of pH and association constants inherent within it. This change over distance, which operates most strongly at the endpoints, permits the system to deliver ions in the way it does.

Charged particles do not easily penetrate membranes, because charged particles are generally not lipid soluble. Although this is generally true, it is not invariably so. If a particle is fairly small and its charge comparatively large, and the membrane is relatively thin, an ion will be dragged through the lipid bi-layer membrane. By arranging the electrical vector in the same direction as the other diffusion vector, this penetration can be greatly improved. This is particularly useful for ions delivered perpendicular to membranes, such as the thin membranes of the nasal conchae in the nose.

Therefore, an antiviral agent of the present invention is ideal for use in therapeutically targeting a viral infection of specific cells and will provide more direct application of an antiviral agent to a targeted site of infection than most conventional antiviral agents. Such advantages allow for the antiviral agent to actually contain a lower dosage of potentially cytotoxic bioactive substance, since a higher percentage of drug is delivered to the target area.

The antiviral agent of the present invention can also be used as a first line of defense against a virus that mutates rapidly, or an engineered virus to which people have not yet developed immunity. The antiviral agent can be administered in conjunction with a vaccine, where available.

The antiviral agent may be any compound that interferes with attachment of the specific virus to be inhibited. For example, one might inhibit respiratory viral infections by using the appropriate molecule resembling its natural attachment site. For coronaviruses such as SARS, one might block Aminopeptidase N, and MHC I docking sites. By blocking the viral attachment sites, interference with the viral life cycle may be accomplished.

Non-ionic agents require an ionizable carrier, which must meet the further requirements of providing for favorable release of the drug at the target site as well as being metabolizable or otherwise easily eliminated physiologically.

In the language of the Teorell-Meyer gradient, the antiviral agent is introduced into a repository compartment and is driven into a recipient compartment, based on the Teorell-Meyer gradient of differing pHs between the two compartments. Use of the antiviral agent entails determining the pH of each compartment, and can be applied to compartments that are adjacent or contiguous, or that are separated only by a thin membrane.

It is expected that a medical or pharmaceutical practitioner of ordinary skill in the art would appreciate the full range of applicability of the invention, as well as specific compounds that interfere with the attachment of each virus sought to be inhibited.

Preparation of the antiviral agent is carried out with an eye towards the type of contiguous recipient compartment system to which this invention applies. Clearly, the skin and the mucosal membranes are also considered to be the most likely recipient compartments. Thus, the preparation of the antiviral agent must be dictated largely by pH differences between the two compartments, although other factors may be present as well. Generally, a difference of at least 0.1 pH units between the compartments is necessary, although the larger the pH difference the faster a bioactive substance will be transported. A pH difference of 2.0 pH units is usually preferred, but a larger difference is possible according to the tolerance of the tissues. Thus, each individual antiviral agent has its own limits based on the practical pH difference between the compartments and each should be prepared according to the desired transport time that makes sense for the system.

The bioactive substance that inhibits viral attachment must also be selected. Here, almost any drug that is in anionic, cationic or ionizable form may be employed. Ionic drugs should be hydrated. Non-ionic drugs may also be used as they can be released from an ionizable carrier such as cyclic carbohydrates and cyclodextrans. The speed of travel of the drug depends on the charge, the atomic or molecular diameter, the molecular weight and the viscosity of the medium in which it travels. The gradient will move any ionic substance with a molecular weight of up to thousands of Daltons.

In the case of a cationic (positively charged) or acid drug, the repository compartment must have an induced pH substantially lower that the recipient compartment. Conversely, for an anionic (negatively charged) or basic drug the repository compartment must have an induced pH higher than the recipient compartment. Thus, the selection of the buffering system for the dosage form is highly significant. The range of buffers employed correspond to the range of pHs found in the human body, the lowest pH presently known is that of the stomach which is about pH 0.1, the highest pH presently known is about 9.0 and is found in the lower intestine. Untraumatized human skin generally has a pH around 5.5-6.0. The buffer or buffer system must last long enough for consumption of the entire dose for complete drug transport to occur.

While the buffers selected must create a pH differential between the compartments of ideally 2.0 pH units or more to cause rapid drug movement, greater or smaller pH differences are not beyond the scope of this invention. When selecting the buffer, physiological considerations must also be taken into account, viz., the amount of pH difference between the dosage buffer and the repository compartment that the tissue of that compartment will tolerate.

For the purpose of this invention, the 20 physiologically accepted amino acids and their congeners (e.g., orotic acid, carnitine, ornitine) are generally preferred. The buffers systems usually contain at least two components: a salt and its correlative acid, or base. Buffers may be single compounds in certain cases, such as solutions of amino acids, Tris®, and other compounds containing both acid and basic groups on the same molecule. A buffering system may be complex, containing several components. It may also contain non-related salts and amino acids or similar zwitterionic compounds.

The buffering agent should be able to reliably buffer at the chosen pH, which may be anywhere within the physiological range, so as to preferably maintain a difference of at least 2 pH units between the repository and recipient compartments, according to tissue tolerance, for the preferred embodiment of the invention, to exert substantial buffering capacity within this range. Preferred buffering agents are the amino acids, hydrogen and dihydrogen phosphates, such as sodium dihydrogen phosphate and mixtures of sodium dihydrogen phosphate with sodium hydrogen phosphate, calcium tetrahydrogen phosphate, citric acid and mixtures of citric acid and its monosodium salt, fumaric acid and its monosodium salt, adipic acid and its monosodium salt, tartaric acid and its monosodium salt, ascorbic acid and its monosodium salt, glutamic acid, aspartic acid, betaine hydrochloride, hydrochlorides of amino acids, such as arginine monohydrochloride and glutamic acid hydrochloride and saccharic acid, and other suitable GRAS ingredients herein incorporated by reference.

As discussed supra, hydro-osmotic pressure, concentration and pH differences between a repository and a recipient compartment form a Teorell-Meyer flux gradient. A Teorell-Meyer flux gradient occurs if there is a two or more compartment unit in which different concentrations, relative charges, and hydro-osmotic pressure exist. There may be one or more ionic substances or electrolytes present, and the method is dependent on total relative force rather than any single element. Thus, the driving force for this dosage form depends on the sum of three vector force components: chemical and electrical force and hydro-osmotic pressure, as comprehensively detailed in U.S. Pat. No. 6,414,033, herein incorporated by reference in its entirety.

To summarize, in the practice of this invention, therefore, the following steps must be observed. To move a positively charged (i.e., acid) ion, drug or pro-drug used as an antiviral agent, the pH of said agent must be lowered below that of the target or destination area for the drug, i.e., the site of the viral infection. Conversely, to move a negatively charged (i.e., basic) drug, the pH of the antiviral agent is raised above that of the site of the viral infection. This movement is osmo-electrophoretic, and the energy is supplied by the Teorell-Meyer concentration gradient. Using this principle as applied to treatment of viral infections, almost any FDA or homeopathically approved bioactive agent may be used. The identification of said agents will be apparent to one of ordinary skill in the art.

As used herein, the term “bioactive substance” or “agent” is identical to the meaning of the term “drug” employed in the 26^{th} Edition of Stedman's Medical Dictionary, viz., “[a] [t]herapeutic agent; any substance, other than food, used in the prevention, diagnosis, alleviation, treatment, or cure of disease.” In addition, for the purposes of the present invention, a bioactive agent may be any substance that affects the activity of a specific cell, bodily organ or function. It may be an organic or inorganic chemical, a biomaterial, etc. Any chemical entity of varying molecular size (both small and large) exhibiting a therapeutic effect in animals and humans and/or used in the diagnosis of any pathological condition, including substances useful for medical imaging such as fluorescent dyes and radioactive isotopes fits the above definition.

### EXAMPLE

Based on the above discussions, an antiviral agent may be formulated to be administered to an infected individual. Said antiviral agent would act, for example, to dock at sites where coronavirus particles attach to cellular receptors.

When the patient inhales deeply, this action drives the virus particles to the bottom of alveoli and sinuses. It is then the therapeutic goal to block viral attachment of particles at sites where the particles would enter the cells. This interferes with the viral life cycle, breaking the pattern of infectivity. This goal might be achieved by means of the present invention: a nasal or oral spray containing a mixture of ions delivers said mixture to its therapeutic target. The ions block the attachment sites, coronavirus receptor aminopeptidase N (CD13), preventing the coronavirus particles from docking on the cell surface and entering the cell.

The foregoing description of the specific embodiments will so fully reveal the general nature of the invention that other can, by applying current knowledge, readily modify and/or adapt for various application such specific embodiments without undue experimentation and without departing from the generic concept. Therefore, such adaptations and modifications should and are intended to be comprehended within the meaning and range of equivalents of the disclosed embodiments.

It is to be understood that the phraseology or terminology employed herein is for the purpose of description and not of limitation. The means and materials for carrying out various disclosed functions may take a variety of alternative forms without departing from the invention.

Thus, the expressions “means to . . . ” and “means for . . . ” as may be found in the specification above and/or in the claims below, followed by a functional statement, are intended to define and cover whatever structural, physical, chemical, or electrical element or structures which may now or in the future exist for carrying out the recited function, whether or nor precisely equivalent to the embodiment or embodiments disclosed in the specification above. It is intended that such expressions be given their broadest interpretation.

As will be apparent to one skilled in the art, various modifications can be made within the scope of the aforesaid description. Such modifications being within the ability of one skilled in the art form a part of the present invention by the appended claims.

What is claimed is:

1. An antiviral agent which delivers a pharmaceutically effective amount of a bioactive substance to a site of virus infection by utilizing a pH difference between a repository site and the site of virus infection that drives delivery of the bioactive substance into the site of virus infection.

2. A method for treating a viral infection, the method comprising:
applying an antiviral agent which delivers a pharmaceutically effective amount of a bioactive substance to a site of virus infection by utilizing a pH difference between a repository site and the site of virus infection that drives delivery of the bioactive substance into the site of virus infection.

3. The method of claim 2, wherein the antiviral agent is a suitably sized, suitably charged amino acid of suitable configuration.

4. The method of claim 2, wherein the virus is a coronavirus.

5. The method of claim 4, wherein the virus is SARS.

6. The method of claim 3, wherein the antiviral agent is at least one of N-acetyl neuraminic acid, neuraminic acid, sialic acid, Zn²⁺, Ca²⁺, Fe²⁺, Fe³⁺, Al³⁺, positively charged amino acids of suitable configuration, and small positively charged peptides.

7. The method of claim 3, wherein the antiviral agent is at least one of bromine, iodine, chlorine, sialic acid, neuraminic acid, N-acetyl neuraminic acid, negatively charged amino acids, and negatively charged peptides.
